(19) United States
(12) Patent Application Publication
(10) Pub. No.: US 2014/0303011 A1
(43) Pub. Date: Oct. 9, 2014

(54) HIGH RESOLUTION CHARACTERIZATION OF BIOLOGICAL MATRICES

(71) Applicants: Daniel Shabacker, Naperville, IL (US); Adam Driks, Chicago, IL (US)

(72) Inventors: Daniel Shabacker, Naperville, IL (US); Adam Driks, Chicago, IL (US)

(73) Assignee: UCHICAGO ARGONNE, LLC, Chicago, IL (US)

(21) Appl. No.: 13/427,203

(22) Filed: Mar. 22, 2012

Related U.S. Application Data
(60) Provisional application No. 61/466,396, filed on Mar. 22, 2011.

(51) Int. Cl. G01N 33/68; G06F 19/20

(52) U.S. Cl. CPC G01N 33/6842; G06F 19/20
USPC 506/8; 506/18

(57) ABSTRACT

The invention provides for a method for distinguishing spore preparation procedures using spore molecular signatures, the method comprising harvesting spores from a sample; extracting molecules from the spores, fractionating the extracted molecules for analysis; generating molecular signatures from the fractionated molecules, and comparing the molecular signatures to a library of molecular signatures.

### PRIORITY

This utility patent application claims the benefits to U.S. Provisional Patent Application No. 61/466,396 filed on Mar. 22, 2011.

### CONTRACTUAL ORIGIN OF THE INVENTION

The United States Government has rights in this invention pursuant to Contract No. DE-AC02-06CH11357 between the United States Government and UChicago Argonne, LLC representing Argonne National Laboratory.

### BACKGROUND OF THE INVENTION

1. Field of the Invention

This invention relates to a method for using molecular signatures to identify preparation methods and any variations or modifications thereof. An example of a specific variation detectable by this invention is differentiating between spores resulting from natural infections and spores produced in the laboratory (as would be the case if the organism was produced for use as a weapon).

2. Background of the Invention

Laboratory technique is both an art and an acquired skill. Like many workers, laboratory technicians who culture bacteria adopt protocols and working habits established at the laboratories in which they trained. Laboratory workers are reluctant to change protocols that have been successful in the past, even when there can be some incentive to do so, because of the risk of unforeseen consequences. Generally, procedural standards initially adopted and learned remain with lab technicians after they leave their initial training environment (i.e. industrial, military, biological weapons production, etc.). In the case of cell culture, and spore culture in particular, aspects of culture protocols that vary among laboratories include adjustments to culture medium, culture treatments for germination prevention, inoculation conditions, and culture duration before harvest. Inasmuch as these parameters can vary, yet still produce usable spores, technicians tend to execute their protocols according to set procedures that they have learned and are reliable. Assessments of these and other variations are good measures for operator skill sets, resources and goals. As such, traceability from any biological agent samples (i.e. bacterial spores, vegetative bacteria, and viruses) to technicians with specific training and skill sets is feasible.

Bacillus anthracis, the causative agent of anthrax, poses significant risk to national security. Detection, decontamination, and identification of the source of such biological weapons enhance national security. The National Research Council has concluded that current science protocols were not definitive in pinpointing spore origins related to the U.S. Mail anthrax attacks of 2001. This has led to the introduction, in February 2011, of the Anthrax Attacks Investigation Act, which will develop a wider scope of evidence than that gathered by current protocols used by the Federal Bureau of Investigation.

Identifying the specific methods used to culture and prepare Bacillus anthracis spores recovered from the field reveals the answers to two critical questions. First, whether the agent is artificially made (i.e. laboratory grown) or naturally occurring. This is central to distinguishing a natural anthrax outbreak from an intentional release. Therefore, distinguishing Bacillus anthracis spores prepared in the laboratory from those produced after a natural infection remains an objective in microbial forensics.

Second, in cases where the Bacillus anthracis spores recovered from the field are not naturally occurring, identifying the specific methods used to culture and prepare the spores reveals the training of the operator who prepared the spores (as well as their resources and goals) and, therefore, specific information about the origin of the spores. Information regarding biological agent culture conditions, preparation, storage and treatments prior to delivery provides critical information for national defense, including but not limited to knowledge of the operator's expertise, resources, and goals. This analysis can be useful for attribution, as culture and preparation methods can be idiosyncratic and, possibly, operator-specific. National defense currently lacks the forensic tools needed for these assessments.

Current microbial forensics techniques rely primarily on genetic analysis to identify genera, species and strains of biological agents. Genetic analyses are most valuable when, after the fact, it turns out that a very obscure strain was used. This is likely to be a rare situation (and was not the case for the anthrax mailings of 2001, which involved the widely used Ames strain). Genetic analysis may be useful when the source of agent is a genetically complex mixed culture and when investigators have access to culture samples from suspects' laboratories. As such, genetic analysis does not usually result in practical forensic information and does not allow the agent to be traced to its operator.

Additionally, genetic analysis focuses on evolution and variation based on isolated growth conditions, which remain very poorly understood. In general, the dynamics of the genetic variation that will take place in a culture cannot be predicted ahead of time. Therefore, while this genetic variation could, potentially, provide a signature, this signature cannot be predicted ahead of time and, if it exists, could vary unpredictably from culture to culture. To obtain insight into environmental conditions related to where and how a specific threat agent was grown solely from genetic analysis would require entering the laboratory where the threat agent was prepared, and obtaining physical evidence for genetic analysis.

Accessibility posses a practical obstacle for options suing genetic analysis as described supra. The first obstacle is gaining access to the operator's laboratory. In cases where authorities can easily enter the lab and obtain all the evidence present, in particular a sample of spore culture, this obstacle is manageable. However, in almost any case that requires access to a laboratory not under United States government law, particularly those involving foreign organizations hostile to the United States (such as terrorists and non-state organizations generally), access to the operator's laboratory is virtually impossible. Thus, spore culture samples cannot be obtained for testing and analysis except by exceptional methods which are usually impractical.

Genotypic analysis of spore cultures allows strain identification, but provides no definitive information about culturing processes. While efforts to understand relationships between genetic variations in cultures and its history are in progress, no method presently exists for this analysis. Even if such methods become available in the near future, there are several practical problems related to genetic analysis, such as access to the operator laboratory, discussed supra. Conjectured future genetic analytic technologies rely on deep sequencing deoxyribonucleic acid (DNA) analysis, which is complex, expensive, and requires highly-skilled technicians and bioinformaticians. As such, this methodology is simply not practical as a routine forensic methodology. At present, no method exists for establishing culture history using genetic analysis.

Currently, molecules harvested from biological threat agents can be detected and analyzed using methods such as 1D sodium dodecyl sulfate polyacrylamide gel electrophoresis (SDS-PAGE), 2D SDS-PAGE, Western blotting, and Mass Spectrometry. Unfortunately, these techniques are either too costly or too delicate to use reliably in the field. Additional drawbacks in using SDS-PAGE are that it is technically challenging to do in a repeatable manner and resource intensive, while capable of capturing only 1 to 5 percent of the molecular information in a substance of interest, such as a threat agent. Also, these current methods do not allow efficient interrogation with common molecular detection reagents.

Robust molecular libraries that attribute molecular signatures to sources are needed. However, molecular libraries have yet to be constructed because the present methods for analysis (SDS-PAGE and related techniques) are too cumbersome for generation of reliable signature libraries.

A need exists in the art for a method to identify changes in protein expression in substances of interest, those changes due to environmental or growth conditions. Substances of interest include biological threat agents (BTAs), and proteins for use in human treatment. The method should provide molecular signatures detailed enough to significantly contribute to attribution, and sufficiently reliable to withstand legal challenges or readily matured to possess such reliability.

### SUMMARY OF INVENTION

An object of the invention is to provide a method to distinguish protein signatures of laboratory-produced macromolecules, specifically biological threat agents such as pathogenic agents, from naturally-produced macromolecules that overcomes many of the drawbacks of the prior art.

Another object of the invention is to provide a method to identify methods of agent culture, preparation, storage, and other modifications prior to agent deployment. A feature of the invention is its combination of 2D-liquid phase fractionation with hybridization matrices. An advantage of the invention is that the combination significantly enhances the technological advantages over 1D and 2D SDS-PAGE and Western blot analysis for characterization of biological threat agents.

Still another object of the invention is to provide a method for identifying a BTA's source and the methods by which it was generated and stored. A feature of the invention is combining matrices of protein libraries with knowledge of which preparation methods generate which protein signatures. This combination provides a means for determining operator background, resources and goals. An advantage is that the system digitally correlates protein signatures of agents gathered in the field with knowledge of preparation methods and, therefore, provides forensic information.

Yet another object of the present invention is to provide a reliable forensic tool that can be used to produce statistical analytics. A feature of the invention is that in generating signature libraries, it will allow protein profiles to be sufficiently consistent for use as a forensic tool. An advantage of the invention is that through developing signature libraries, confounding signatures can be identified and characterized and subsequently removed from the analysis. An additional feature of the invention is that the data output can directly interface with standard statistical programs so as to identify the method used for the growth and preparation of biological agents. An advantage of the invention is that the automated statistical analysis narrows the pool of potential suspects to withstand legal scrutiny without having to validate methodology.

Briefly, the invention provides a method for obtaining information about any macromolecule preparation, the method comprising harvesting spores from a sample; extracting molecules from the spores, fractionating the extracted molecules, generating molecular (including but not limited to proteomic) signatures from the fractionated molecules, generating molecular (including but not limited to proteomic) signatures from said fractions, and comparing the molecular signatures to a library of molecular or protein signatures. In an alternate embodiment of the invention, instead of harvesting molecules from the spores, molecules are harvested from the culture medium in which spores were cultured (spent medium).

The invention also provides a method for identifying the source, method of preparation, treatment after preparation and before storage, and the method of storage of a spore of a target bacterium, the method comprising comparing molecular signatures of the spores of a well-known strain of the target bacteria with a molecular signature of the spores of a natural strain of the target bacteria to determine a strain variation pattern; and comparing the strain variation pattern to molecular signatures of the spore of the target bacteria to determine features of the molecular signatures of the target bacteria not caused by strain variation. The invented method characterizes variations in protein expression due solely to strain isolates and eliminates from analysis features of the proteomic signatures not caused by strain variation.

Also provided is a device for detecting the source of a biological substance, the device comprising a first instrument to harvest samples; a second instrument to extract macromolecules from the harvested samples; a third instrument to fractionate the extracted macromolecules; a fourth instrument and software to generate a proteomic signature from said fractionated molecules; and a proteomic signature library comprising fingerprints of preparation methods and culture histories, the signature library used to analyze the generated proteomic signature.

The invention provides a method for identifying a biomarker, the method comprising obtaining proteins from a target organism; separating the proteins into a plurality of fractions; isolating the fractions in separate reaction chambers, wherein each of the reaction chambers are arranged in a two-dimensional matrix according to isoelectric point values and hydrophobicity values; contacting each of the fractions with a cocktail of reagents, wherein each of said reagents is capable of binding to less than all of the proteins; and determining which reagents indicate the presence of protein in specific reaction chambers in the matrix.

### BRIEF DESCRIPTION OF DRAWING

The invention, together with the above and other objects and advantages, will be best understood from the following detailed description of the preferred embodiment of the invention shown in the accompanying drawings, wherein:

FIG. 1 is a micrograph of a biological threat agent spore, specifically a Bacillus anthracis spore;

FIG. 2 is a schematic depiction of the invention;

FIG. 3 is a depiction of a biochip, an image from a biochip scanner, and a heat map generated from the information on a biochip;

FIG. 4 is a depiction of staining patterns from hierarchical clustering;

FIG. 5 is a depiction of the difference in molecular signatures when comparing spores grown in solid medium versus spores grown in liquid medium;

FIGS. 6A-C are a series of heat maps and UV absorbance graphs depicting relative amounts of protein from anthrax cultured in different media, in accordance with features of the present invention; and

FIGS. 7A-C are a series of heat maps and UV absorbance graphs depicting relative amounts of protein from anthrax cultured in different media, in accordance with features of the present invention.

### DETAILED DESCRIPTION OF THE INVENTION

The foregoing summary, as well as the following detailed description of certain embodiments of the present invention, will be better understood when read in conjunction with the appended drawings.

As used herein, an element or step recited in the singular and proceeded with the word “a” or “an” should be understood as not excluding plural said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

The invention provides a method for obtaining information of biological threat agents, including their methods of culture of biological threat agents, their treatment after culture, storage after growth, and treatment during storage and treatment after storage. This information provides a means for determining the source of the BATs, including the location of the source, the skill of the operator, and the identity of the regime generating the BATs. Advantages of this invented method include using modest sample quantities (less than about 5 μl of sample), rapid analysis (completed within minutes), and off-the-shelf technology all within a very low cost frame work and a low tech environment. Also, the invention is tolerant of environmental contamination, including but not limited to soil particles, ambient trace materials such as environmental pollutions endemic to certain geographic regions, building materials, additives used in the preparation of BATs and the general preparation of bacteria.

The invention is also tolerant of interferents designated by the U.S. Department of Defense Critical Reagents Program such as, but not limited to, burning vegetation, burning diesel, malathion, Aspergillus niger, clay soil, sage pollen, burning fog oil, burning rubber, HC smoke gunshot, Vero cell supernatant, sandy soil, loamy soil, bovine serum albumin, nutrient broth, BHI broth, G media with trace minerals, Brucella broth, TSB broth, PBS, burning rags, Tween 80, and combinations thereof. This tolerance is for at least the following five reasons: First, large amounts of data are collected for each proteome; as such, effects on a fraction of collected data will likely have little consequence to overall analysis. Second, fractionation methods used will exclude most of the contaminating molecules listed above from downstream analysis. Third, use of binding reagents, such as lectins, antibodies, tagged aptamers of any molecular composition, or any other ligands that bind covalently or noncovalently to the fractionated molecules, will further reduce detection of irrelevant molecules and also reduce any negative effects of those interferents on detection of the targeted molecules. Fourth, the application of statistical analysis to the signature library data will enable identification of those proteomic fractions that are most relevant to forensic analysis. As a result, much environmental contamination will never be included in the data interrogation.

Finally, the creation of well established libraries enables the development of modular (i.e., portable) kits, such as field kits. The kits compare a harvested sample's signature to existing signature libraries for an in situ and/or immediate determination of growth culture used, and preparation and storage methods utilized. Therefore, the method enables the determination of covert lab location (e.g., bydistinguishing spores produced in a friendly laboratory from those produced in an enemy laboratory).

The invented method for identifying sources of BTAs has the following properties:
Fractionation processes that are efficient and reproducible;
Physically robust;
Inexpensive;
Highly sensitive;
Possess high resolution;
Transportable;
Easily reproduced;
Easily implemented;
Is an alternative to mass spectrometry; and
Capable of generating interactive signatures with standard biochemical reagents.

The invention provides a robust method for generating molecular signatures with sufficient detail to support forensic attribution beyond a reasonable doubt. The method identifies any variations in macromolecular composition whether from spores, non-spores, viruses, or biological toxins, those variations which are a function of agent preparation, storage, other treatments, or ubiquitous substances in the ambient environment.

Variations in agent preparation include but are not limited to, variations in culturing temperature, culture inoculation conditions, culture medium composition, use of a fermentor rather than conventional flasks, inclusion of specialized molecules and/or other reagents as stabilizers, dispersants or for other purposes, durations of culturing, duration of storage, storage conditions (temperature, humidity, buffer type), and purification methods. The category of “preparation” includes the effects, over varying periods of time, of the environment(s) in which the agent is stored after production (including containment vessels, partially or fully assembled weapons and/or deployment devices (such as overt or covert sprayers), and the environments in which the agent is deployed. The “environment” in which the agent is deployed includes the environment(s) in which the agent is in contact after the agent is released by the weapon (or an individual dispersing the agent, or whatever method is used to place the agent in the environment and/or in contact with victims, whether targeting humans, agricultural animals or plants, or infrastructure (as in plastic-degrading, or metal corroding bacteria).

Even modest process variations (such as the presence of trace airborne molecules) create changes in composition readily detectable by the invented method. This results in a high likelihood in distinguishing among signatures of different laboratory protocols and between spores formed in a laboratory as opposed to in nature as shown in Characterization of spores of Bacillus subtilis which lack dipicolinic acid by Paidhungat, M., B. Setlow, A. Driks, and P. Setlow, (J. Bacteriol., 2000, 182:5505-5512) and Comparison of the properties of Bacillus subtilis spores made in liquid or on agar plates by Rose, R., B. Setlow, A. Monroe, M. Mallozzi, A. Driks, and P. Setlow (J. Appl. Microbiol, 2007, 103:691-699), both of which are incorporated by reference. For example, these studies show how signatures distinguish between spores cultured on solid versus liquid medium. This is relevant because operators creating biological weapons are likely to use solid medium inasmuch as solid media-based protocols can be conducted easily under rogue conditions, such as in a cave; whereas liquid medium protocols require more sophisticated equipment and precise handling.

The invention provides high reproducibility and achieves very high resolution fractionation of the protein proteome. It is highly sensitive compared to existing molecular analytic methods used to analyze the molecular compositions of cells, and efficient in several ways, including the use of a stabilized, highly reproducible fractionation method, incorporation of rapid and semiautomated data collection, and elimination of the need for mass spectrometry. The method enables the immediate searching for signatures of laboratory cultures, without the need for further theoretical studies to support the methodology, given its use of well established basic science on the sensitivity of the coat protein proteome to culture conditions. Furthermore, this invention is capable of readily distinguishing between two relatively similar but distinct preparation methods, so as to identify the skills and resources of the operator, among other critical information.

As a control, the proteomic signature from at least one natural strain is compared with the proteomic signature of that strain produced under laboratory-culture. In an embodiment of the invented method, proteomic signature from cells collected from a natural outbreak (such as Bacillus anthracis spores collected from an outbreak in a game preserve) is compared with the proteomic signature of cells of that same strain cultured in the laboratory. This provides a means for verifying that signature differences detected between natural and laboratory-cultured spores are not due solely to genetic differences among strains. Specifically, the method provides a means for identifying the portion of a signature that distinguishes natural from laboratory-cultured cells.

The invention is best illustrated using B. anthracis spores as an example. However, the discussion infra of B. anthracis should not be construed as limiting the invention to only this bacterium or to any class, taxon or other grouping of toxins, viruses or bacteria (such as spores, or bacteria that can produce spores). Other pathogenic or non-pathogenic bacteria and viruses are noteworthy security risks. For example, nonpathogenic bacteria similar enough to a threat agent may be used by rogue laboratories to encumber investigations. Forensic analysis of these nonpathogenic interferent bacteria has important value to an investigation, similar to an analysis of a pathogen. The method disclosed in this present invention can be used on any organism or product (e.g. toxin) of an organism that can be cultured in a laboratory. Suitable organisms include, but are not limited to, B. anthracis, any Bacillus cereus-group bacteria and any spore forming organism from the Bacilliaceae or Clostridiaceae families are also suitable targets. Relevant bacteria include, but not limited to, all bacteria on the Centers for Disease Control and Prevention Bioterrorism Agents/Diseases category A, B and C lists, located at the Centers for Disease Control, Atlanta, Ga.

### Matrix Protein

### Preparation Detail

In an embodiment of the invented method, laboratory-cultured spores are generated from attenuated Sterne strain (strain 34F2) using three conventional laboratory protocols and two purification methods. An embodiment of these methods is found in Giorno R., et al., Microbiology, April, 2009, 155 (Pt 4):1133-45, which is incorporated by reference. The Sterne strain 34F2 is commonly available from academic laboratories across the world; such laboratories including, but not limited to, the Olaf Schneewind Laboratory at the University of Chicago in Chicago, Ill. and the Philip Hanna laboratory at the University of Michigan Medical School. The following laboratory protocols among others are suitable: growth to exhaustion of the culture medium in liquid Difco sporulation medium, liquid Leighton-Doi medium, and solid Difco sporulation medium.

Field strains are used to establish a signature for naturally-occurring spores, and therefore represent natural infections. Inasmuch as endemic areas across west Texas have annual outbreaks with at least two different B. anthracis lineages present, naturally-produced spores are collected from diagnostic samples of wildlife and/or livestock outbreaks from these areas. Animal samples (such as nasal turbinates, bone fragments for dried marrow, or pieces of hide) and soil samples associated with carcasses are used to detect both the genotype and the presence of B. anthracis in field samples using culture, Polymerase Chain Reaction (PCR), and subtyping analyses.

In the present invention, composition of the outermost protein shells encasing a spore is analyzed, inasmuch as composition of these shells varies with spore preparation method.

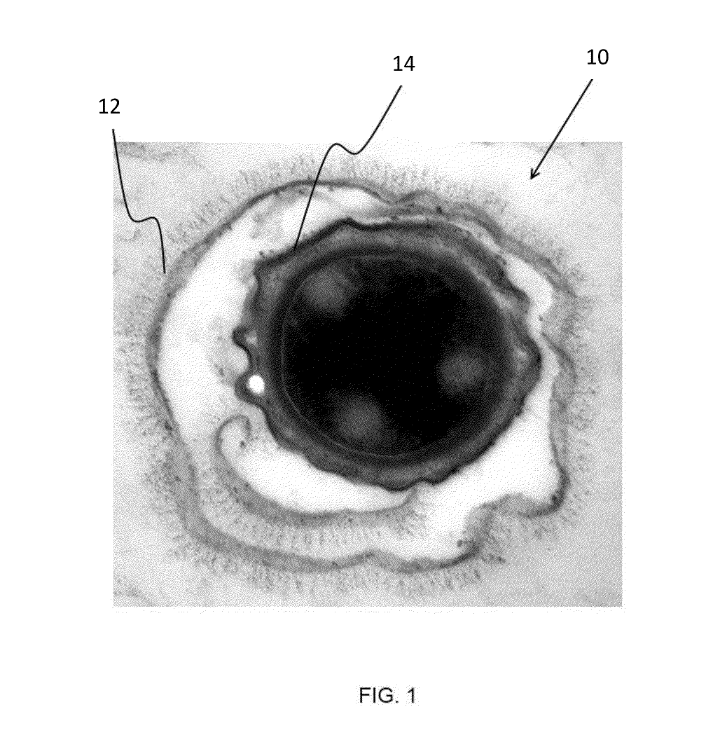

FIG. 1 is a micrograph of a B. anthracis spore, the spore designated as numeral 10. The spore is encased in two substantially continuous protein shells, a coat 14 and an exosporium 12. An advantage of using the coat 14 as a major part of the signature is that it contains more than 70 proteins, many of which are sensitive to spore culture and preparation conditions as noted supra. Such preparation conditions include but are not limited to medium composition, temperature, duration of culture, and storage conditions of the spores as discussed in Paidhungat et al. and Rose et al., this reference introduced supra.

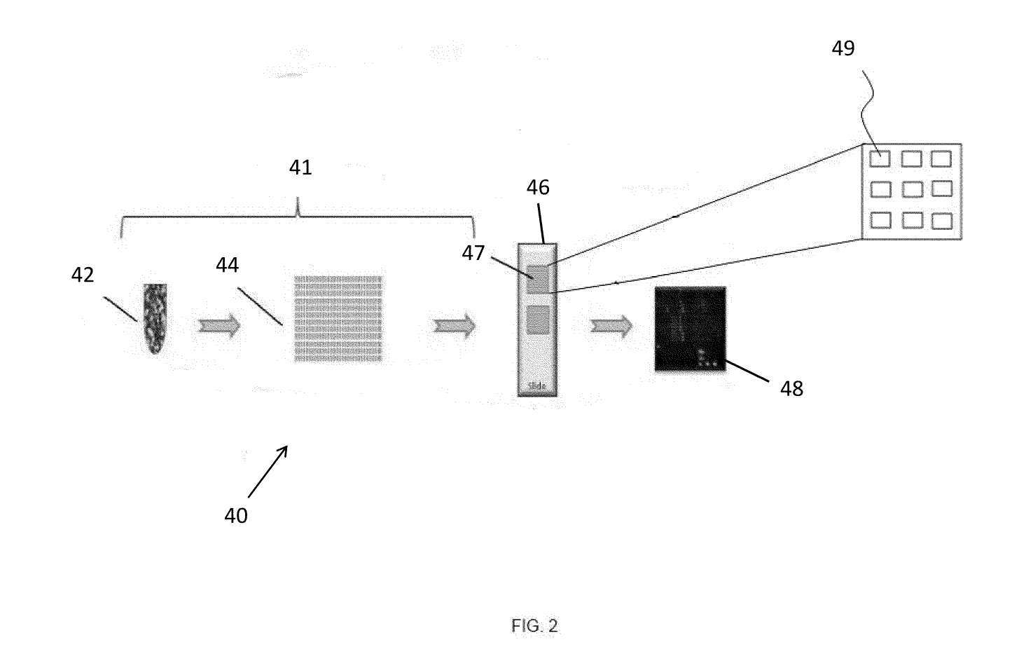

Target protein macromolecules are chemically extracted from the coat 14 of spores using routine approaches, found in Functional analysis of the Bacillus subtilis morphogenetic spore coat protein CotE by Little, S., and A. Driks (Mol. Microbiol., 2001, 42:1107-1120), which is incorporated by reference. This extraction, labeled as element 42 in FIG. 2, is then subjected to fractionation, as detailed below.

FIG. 2 is a schematic depiction of the invented analysis process 40 for the extracted protein macromolecule. The process 40 combines 2D-liquid phase fractionation with biochip technology. First, the protein macromolecule extractant 42, is subjected to a macromolecule fractionation system 41. Several automated systems are commercially available, for example the ProteoSep 2D Fractionation device from Eprogren (Darien, Ill.). The fraction system sorts molecules, such as proteins by their physicochemical properties 44. One such physicochemical property is Isoelectric point (pp. (pl is the pH where a particular molecule or surface carries no net electrical charge.) Minor differences in protein composition can be differentiated by pl. Fractionation occurs specifically through an ampholytic gradient. Proteins with different pl properties are separated through this gradient and into fractions. Other physiochemical properties for use as fractionation parameters include, hydrophobicity, size, bulk charge, zeta potential, ability to bind to anionic and/or cationic column matricies, electrophoresis by capilary methods, fractionation by solid state methods including the Agilent system, passage through a spectrometer, and combinations thereof.

Following fractionation, each fraction is transported from its reaction chamber and deposited in a cell on a matrix. (In an embodiment of the invention, first following fractionation and before deposition, the fractions are buffer exchanged into array buffer removing acetonitrile and trifluoroacetic acid.) In an embodiment of the invention the matrix is supported by a substrate 46. Each fraction is deposited onto the substrate via a robotic system. The location of each fraction in the matrix is recorded along with that fraction's aforementioned physicochemical properties. This fraction transport and deposition process is enabled in a myriad of ways. In an embodiment of the invented method, correlation occurs via microarray deposition software, as is commercially available. For example, suitable tracking software and hardware is GenePix available through Molecular Devices, LLC (Sunnyvale, Calif.) Alternatively, transfer and deposition of each fraction from its reaction chamber to a cell on a matrix occurs so that the fractionation ordering generated by the two dimensional sorting is maintained.

Each cell or spot on the matrix defines a reaction venue such as a spot, gel, or chamber, but the venue does not necessarily need to be of appreciable volume. Rather, a location for deposition is all that is necessary. However, a more typical substrate, commonly referred to as a “chip” 46, does comprise a matrix of individual reaction chambers (such as polyacrylamide pads), each of the chambers discrete from each other so as to be chemically isolated from each other. Methods for making the chip are commercially known. U.S. Pat. No. 5,851,772, incorporated by reference, provides one method for preparation of these matrices. Other methods of adsorption involve nitrocellulose membranes, which facilitates passive adsorption of fractionated molecules. However, for staining aldehyde or carboxyl chips, adsorption would occur through covalent bonds. Protein levels and identification are determined using the methodology discussed in the Giorno R. et al. reference introduced supra.

The now-loaded chip is contacted with reagents that identify protein, polysaccharides and/or other biological molecules and their relative abundance. Relative abundance of the protein, polysaccharides and other biological molecules can be determined using methods selected from a group consisting of, but not limited to ultraviolet absorbance, mass spectrometry reacting said fractionated molecules with appropriate fluorescence-tagged detection molecules (including, as an example, fluorescently tagged lectins), and reacting said fractionated molecules with small molecule dyes.

Reagents capable of distinguishing these molecules from among those in a preparation are suitable. For example, reagents are utilized which are capable of distinguishing one protein from another in a target organism. Reagents are also utilized which are capable of detecting the presence or absence of a protein or substance between different feedstocks, those feedstocks being naturally occurring or artificially engineered. Suitable reagents include, but are not limited to antibodies, biotin, and agglutinating proteins such as lectins, that identify subclasses of polysaccharides. Surprisingly and unexpectedly, the inventors found that streptavidin is a suitable reagent to react with adsorbed fractionated proteins, even in the absence of biotin labeled lectins. Thus, in an embodiment of the invention, fluorescently labeled streptavidin is first contacted with fragmented proteins adsorbed to the slide substrate such as a microchip. This adsorption generates an interactive signature capable of differentiating growth conditions.

The chip can also be contacted with nonspecific protein dyes that detect all proteins bound to the chip as described in “Protein array methods for undefined protein content, manufacturing quality control, and performance validation” by D. Schabacker, I. Stefanovska, I. Gavin, C. Pedrak, and D. Chandler (Anal. Biochemistry 2006, 359:84-93), and U.S. Pat. No. 7,799,570, both of which are incorporated by reference.

These identifying steps provide a means for detecting the types, amounts, concentrations and/or levels of proteins adsorbed to each chamber or cell (each chamber defining an individual cell of the matrix) of the chip.

### Fractionation Detail

The automated fractionation process 41, involves two biochemical separation methods performed in succession: chromatofocusing and reverse-phase HPLC. The interaction of the two technologies provides a resolution synergy not anticipated when the technologies are employed remote from each other. For example, conventional spore fractionation by SDS-PAGE yields a signature. However, it is limited in the information generated, and too technically difficult to achieve, for practical forensics. However, the invented automated high-resolution fractionation system 41 overcomes inherent PAGE technology limitations on the fractions it generates, by first extracting those fractions 44, and then combining the extracted fractions with control elements onto a 1 cm² array 46. This results in a degree of resolution, automation, reproducibility and physical stability that is not possible with either 1-dimensional or 2-dimensional PAGE technology.

In an embodiment of the invention, high resolution sorting is enabled when extracted macromolecules are fractionated and separated into one thousand tubes by their physicochemical properties, such as isoelectric point, discussed supra. This sorting process provides a means for fractionated proteins to maintain their biological reactivity, resulting in fractions being preserved for later analysis. As such, this combination results in both high resolution and repeatability for result verification.

The automated deposition phase consists of adsorption of thousands of isolated fractions, each fraction robotically deposited onto a chamber or cell 49, defining a matrix 47. This results in a protein chip 46 used for analysis. Each cell 49 of the biochip is adapted to receive and maintain, in a stable platform, a specific protein or protein fraction, thereby ensuring high density and fidelity of biochemical signature information when the chip is analyzed as a whole.

A chip-reader is used to quantify the binding of the fractionated protein to the chip by generating an image of a heat map 48. The heat map can be generated by any conventional heat map software, such as, but is not limited to, Cluster Version 2.11, publically available from the University of California Berkeley's Eisen Laboratory. Conventional statistical analysis software is used for analysis, such software including, but not limited to Prediction Analysis for Micrarrays (PAM) Version 2.1, publically available from Stanford University's Health Research and Policy Center, Stanford, Calif.

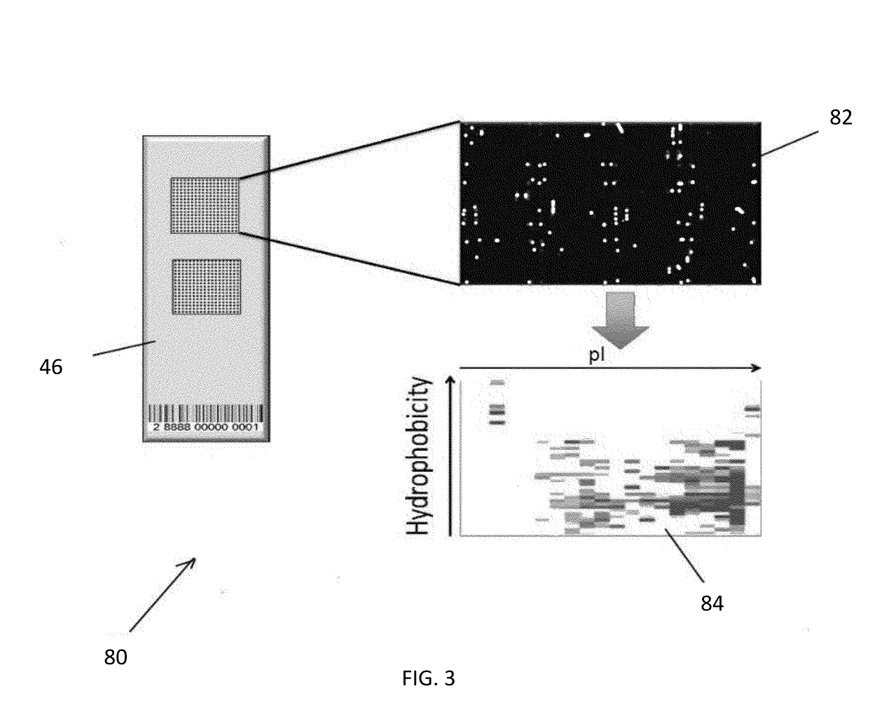

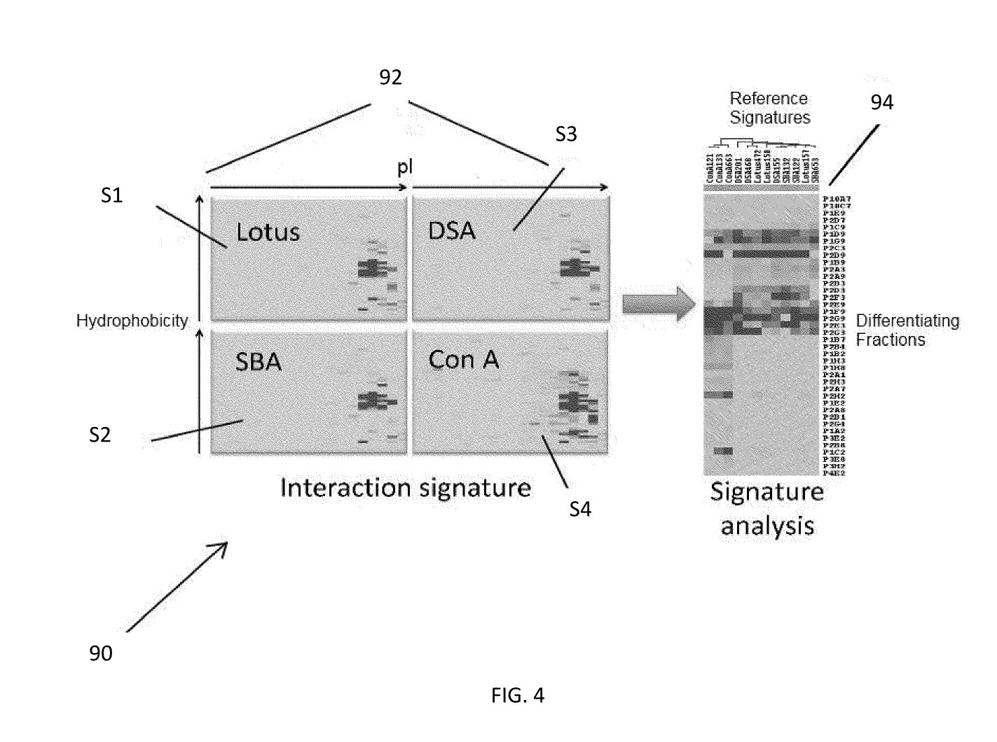

The automated analysis phase depicted in FIG. 2 is better exemplified using FIG. 3 and FIG. 4.

FIG. 3 illustrates a process 80 for creating a heat map used to generate a molecular signature for comparative analysis used to identify various growth conditions and/or preparation methods. First, an image 82 is generated from a biochip scanner, wherein the scanner scans the biochip 46 created from the fractionation phase. Quantification of protein intensities is enabled by the aforementioned reagent interaction with the sequestered fraction in each cell. In embodiment of the method, the entire chip is stained initially to quantify the presence of each protein, as opposed to identification of specific individual proteins. This set of quantified protein intensities also aids in determining the molecular signature, as this information can itself be treated as part of the signature.

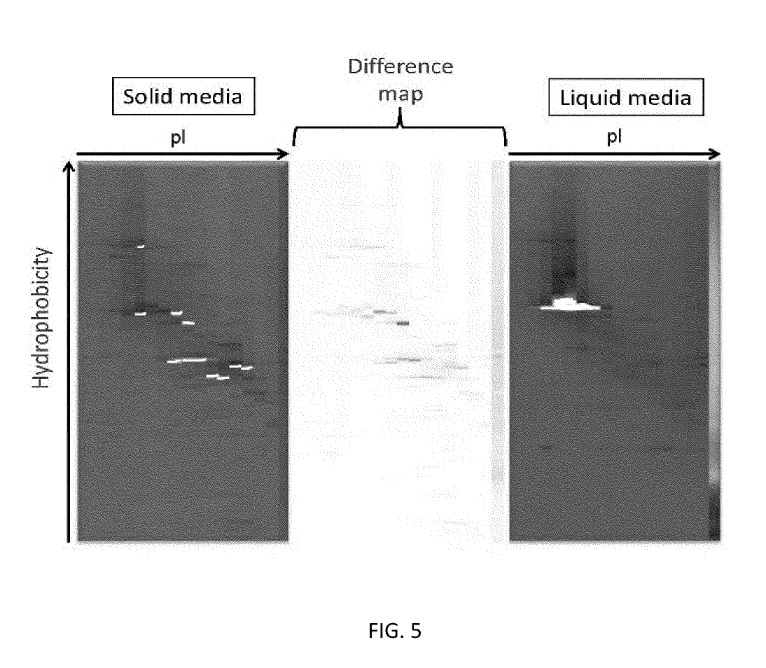

Using conventional statistical analysis software, a heat map 84 is then generated. Heat maps (see FIG. 4 and FIG. 5) provide qualitative analysis in that they are capable of identifying proteins present in samples, as well as quantitative information regarding the amount of a specific protein present.

### EXAMPLE 1

FIG. 4 is a schematic depiction 90 of the application of four different sugar-binding proteins S1-S4 to a single sample 92. The results of this application are patterns that can be analyzed by hierarchical clustering 94. An example of this cluster 94 is an overlay of the four lectin binding patterns which indicate glycosylated molecules. In an embodiment of the invention, cluster analysis is automated. Tags are chosen based on their known properties of binding to a particular macromolecule, for example, Lotus tetragonolobus (Lotus), Dantura stramonium (DSA), soybean agglutinin (SBA) and Concanavalin A (ConA) are lectins that have affinity to fractionated B. anthracis spore proteins.

Determination of spore preparation and growth specifics is best exemplified in FIG. 5 where a noticeable difference in proteome patterns are observed between spores grown in solid medium versus spores grown in liquid medium.

### EXAMPLE 2

Bacillis anthracis spores were prepared using either solid brain heart infusion medium (solid BHI) or liquid Difco sporulation medium (liquid DSM), after which spore coat proteins were purified from the spores. Three replicate cultures were prepared for each growth condition. The six preparations were each separated by PF2D, where, in the first dimension the proteins are separated by pl, and then, in the second dimension, each pl fraction is separated by reverse-phase chromatography, resulting in 768 liquid protein fractions. The fractions were arrayed on PATH protein microarray slides (Grace Bio-Labs) using a QArray2 robot (Genetix). Lectin-binding assays were performed on the microarrays using biotin conjugated lectins (EY Laboratories, Inc.) (Table I). Lectin binding was detected using streptavidin with a fluorescent tag, Alexa Fluor 647.

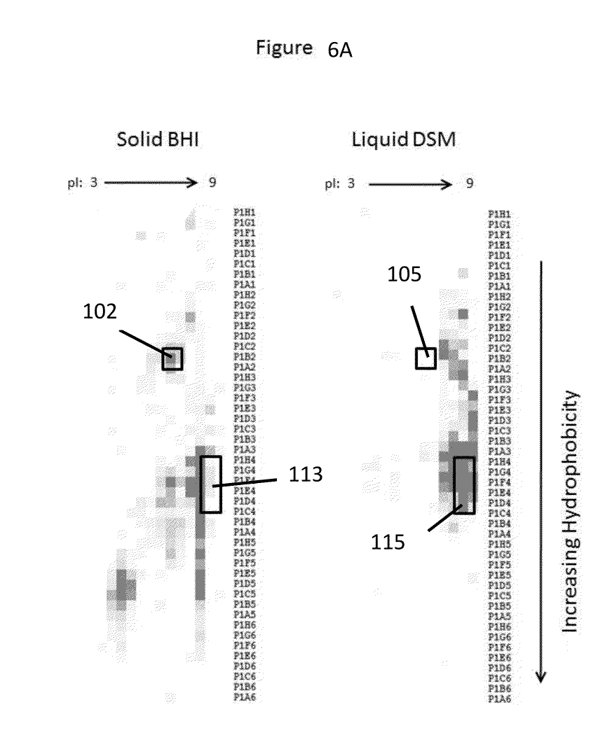

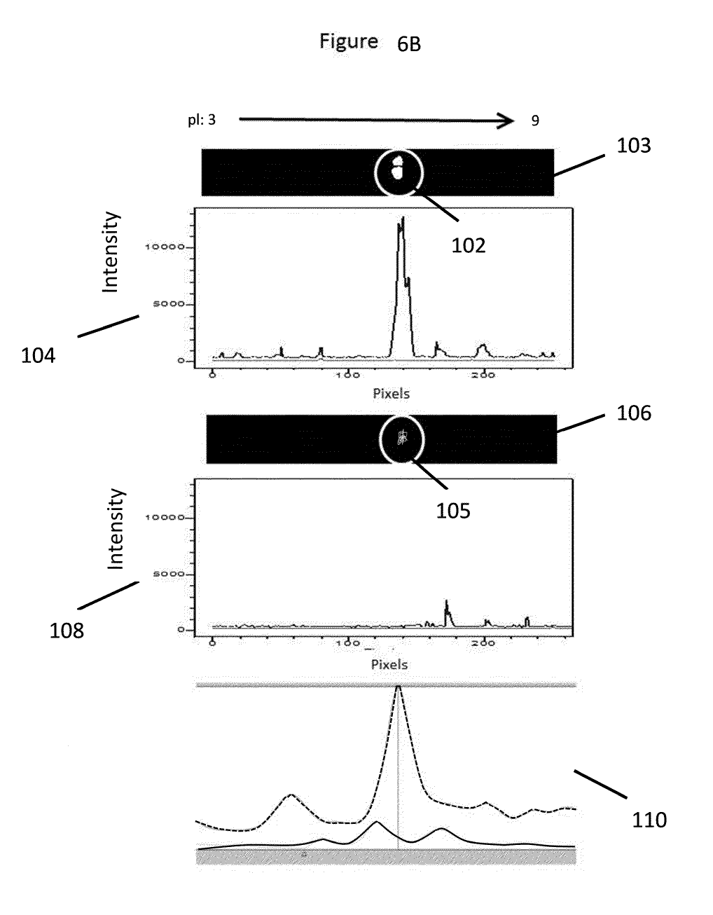

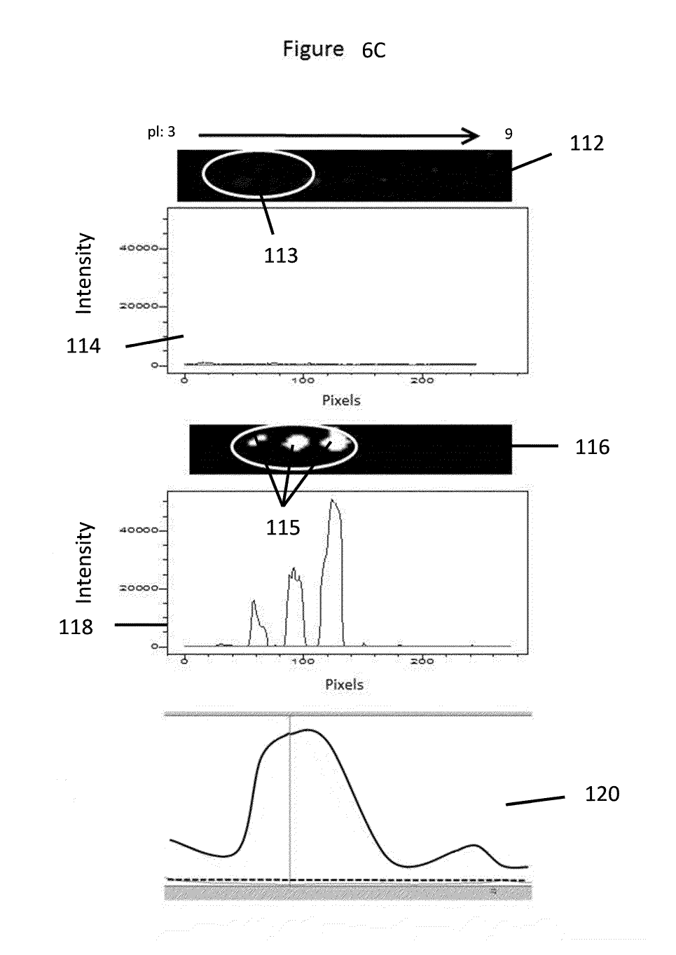

FIG. 6A displays binding of the lectin UEA-I to protein fractions in the arrays displayed as heat maps. The intensities of grey and dark regions indicates the magnitude of the fluorescence signals. The solid BHI heat map (left) and the liquid DSM heat map (right) are each from trial one of the three culture replicates. (The heat maps of the replicate culture preparations look similar; data not shown.) The data for each heat map represents the average of four replicate UEA-I binding assays. The upper squares and the lower rectangles in the heat maps show protein fractions that give different lectin-binding signatures for the two different growth conditions. These differences also appear in heat maps of MAA, SNA, GSI and PNA lectin binding (data not shown).

TABLE 1
Lectins Used to Probe Protein Microarrays

| Lectin | Carbohydrate specificity |
|---|---|
| UEA-I: Ulex europaeus | α-L-fucose |
| PNA: Arachis hypogaea | terminal β-galactose |
| SNA-I: Sambucus nigra | ANA(Neu5Ac α(2,6)Gal/GalNAc) |
| MAA: Maackia amurensis | sialic acid α(2,3) galactose |
| GS-I: Griffonia simplicifolia | melibiose, α-D-galactose |

FIG. 6B provides graphic representations of protein fractions highlighted by the heat maps depicted in FIG. 6A. Element 103 repeats a row of eight protein fractions on the UEA-I microarray (eight of 768 fractions) for the solid BHI growth conditions. The fraction 102 that is circled corresponds to the upper square 102 in the solid BHI heat map. A graphical representation 104 of the intensities of the eight protein fractions is also shown.

Similar to the solid culture medium data, a row 106 of eight protein fractions on the UEA-I microarray (eight of 768 fractions) for the liquid DSM growth conditions is depicted. The fraction 105 that is circled corresponds to the upper square 105 in FIG. 6A, liquid DSM. A graphic depiction 108 of the intensities of the eight protein fractions from spores cultured in DSM is also provided.

UV absorbance spectra 110 of the solid BHI-derived protein fraction (dashed line) and liquid-derived DSM protein fraction (solid line) circled in protein rows 103 and 106 is provided. The UV spectra shows that there was substantially more total protein in the solid BHI preparations. Therefore, the absence of an absorbance peak for the liquid DSM samples provides a means for indicating that the positive signal 102 is a useful component of the signature and not a concentration effect.

FIG. 6C displays a row 112 of eight protein fractions on the UEA-I microarray for the solid BHI growth conditions. The fractions 113 that are circled correspond to three of the fractions in the bottom rectangle seen in FIG. 6A, for solid media BHI. A graphical representation 114 of the intensities of the eight protein fractions in the protein row 112 is provided.

FIG. 6C also depicts a row 116 of eight protein fractions on the UEA-I microarray for the liquid DSM growth conditions. The fractions 115 that are circled correspond to three of the fractions in the bottom rectangle of 6A, i.e, the liquid DSM heat map. Since there is a lot more total protein in the solid BHI samples, these positive protein fractions in the liquid DSM samples are significant. There would be a much greater difference between liquid and solid if the signals were normalized for total protein. Spores grown in the liquid DSM medium produce proteins with higher pl's.

A graphical representation 118 of the intensities of the row 116 of the eight protein fractions is also provided. Lastly, UV absorbance spectra 120 of the liquid DSM protein fractions, 116 (solid line) and solid BHI protein fractions, 112 (dashed line). There is only one broad peak for the three protein fractions, which indicates the same protein eluting from the reverse phase column in consecutive fractions.

### EXAMPLE 3

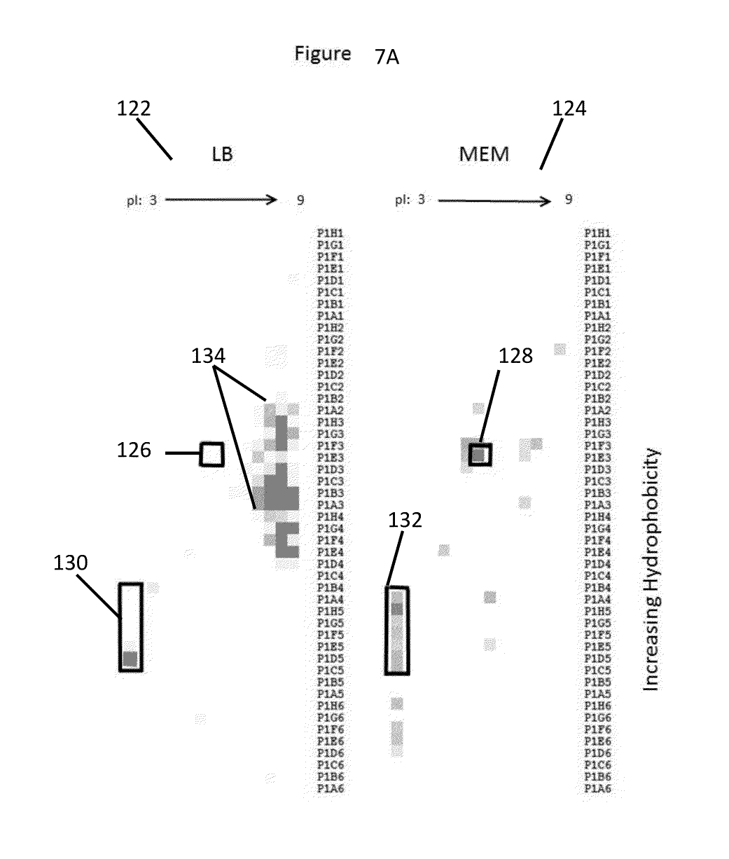

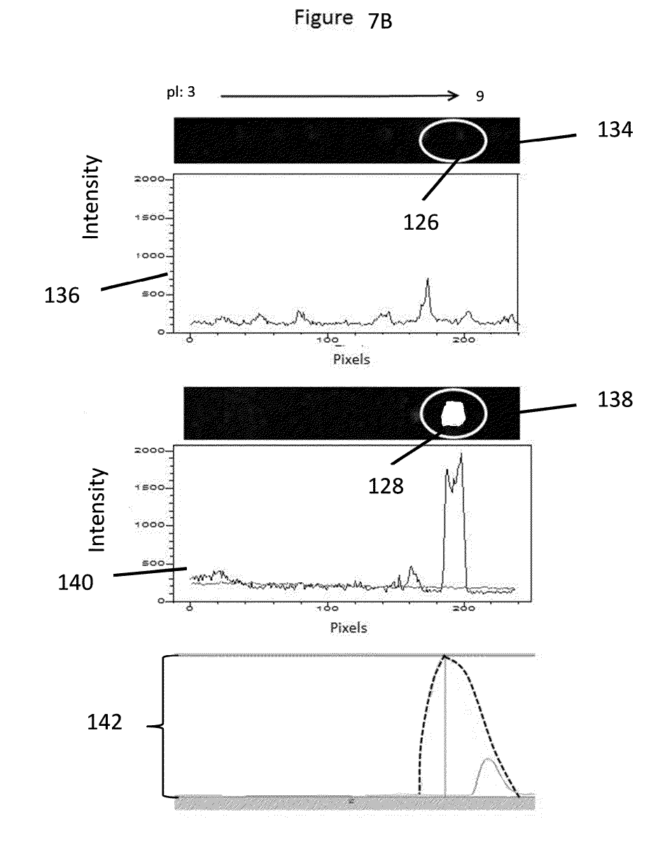

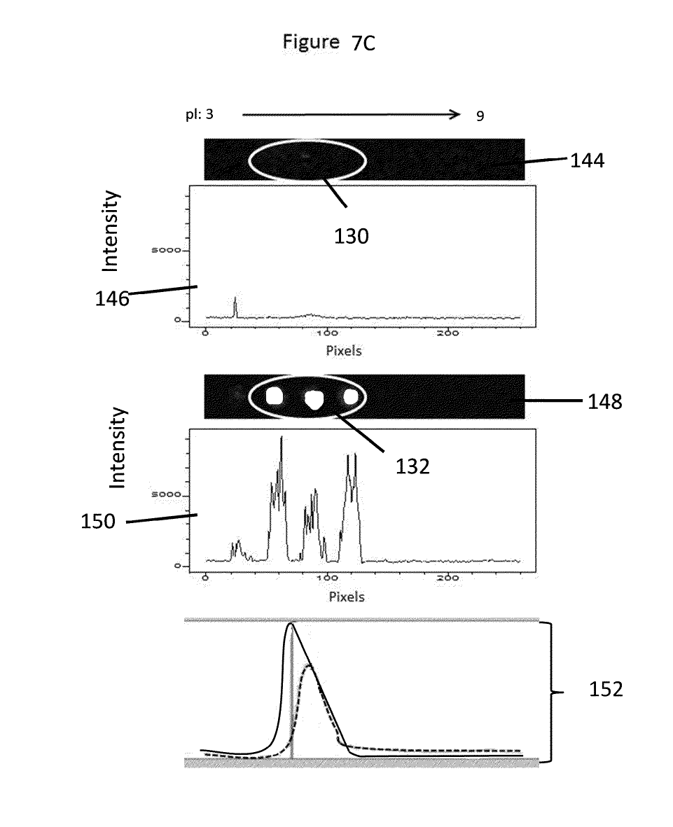

FIG. 7 presents differentiation data related to Escherichia coli. Two cultures of E. coli were grown: one in Luria-Bertani broth (LB) and one in a minimal medium (MEM). The cells were harvested, washed and lysed to harvest the total protein. The proteins from each growth condition were separated by PF2D, where in the first dimension the proteins are separated by pl, and then each pl fraction is separated by reverse-phase chromatography in the second dimension, resulting in 768 liquid protein fractions. The fractions were arrayed on PATH protein microarray slides (Grace Bio-Labs) using a QArray2 robot (Genetix). Anti-E. coli immunoassays and lectin-binding assays were performed on the microarrays using biotin conjugated lectins (EY Laboratories, Inc.) (Table I) and anti-E. coli polyclonal antibody, biotin conjugate (Thermo Scientific). Binding was detected using streptavidin with commercially available fluorescent dyes, such as Alexa Fluor® brand tags, available from Molecular Probes (Eugene, Oreg.).

FIG. 7A shows the binding of the lectin, MAA, (Table 1) to protein fractions in the microarrays, where binding is displayed as heat maps, 122, 124. Dark regions indicate the magnitude of the fluorescence. The LB heat map 122 and the MEM heat map 124 were created from microarray fluorescence data for one replicate each. The upper squares 126, 128 and the lower rectangles 130, 132 in the heat maps show protein fractions that give different MAA binding signatures for the two growth conditions. Heat maps for lectins MAA, SNA, GSI and PNA all show the same positive signal in the upper box 128 for the MEM growth condition, but the LB data 126 is noisy (data not shown). It was also observed that growth 122 in LB produces much higher total protein than growth in minimal media.

FIG. 7B provides graphic representations of protein fractions highlighted by the heat maps depicted in FIG. 7A. Element 134 repeats a row of eight protein fractions (eight of 768 fractions) on the MAA-reacted microarray 122 for the LB growth condition. The fraction that is circled corresponds to the upper square 126. A graphical representation 136 of the intensities of the eight protein fractions is also shown.

Also shown is a row 138 of eight protein fractions (eight of 768) on the reacted microarray for the MEM growth condition, as depicted in the MEM heat map 124. The fraction that is circled corresponds to the upper square 128 in the map 124. Even though there is much less protein under these growth conditions, the invented method still yielded a positive response for this E. coli fraction. A graphical depiction 140 of the intensities of the eight protein fractions seen in the protein fraction row 138 is also provided.

Lastly provided is UV absorbance spectra 142 of the LB medium-cultured protein fraction 126 (solid line) and MEM protein fraction 128 (dotted line), confirming the differential protein expression.

FIG. 7C provides protein fraction data for other isolated proteins 130, 132 on the microarrays 122, 124. A row 144 of eight protein fractions on the MAA microarray for the LB growth condition. The fractions 130 that are circled correspond to fractions in the bottom rectangle of the LB heat map 122. A graphical representation 146 of the intensities of the eight protein fraction row 144 is also provided.

A row 148 of eight protein fractions on the MAA-reacted microarray for the MEM growth condition are depicted. The fractions 132 that are circled correspond to three of the fractions in the bottom rectangle of the MEM heat map 124. While there is more total protein in the samples grown in relatively richer medium, e.g., (LB) medium, the appearance of spots in the MEM maps indicates that the invented method provides a means for detecting E. coli grown in MEM. There would be a much greater difference between the signals for samples cultured in LB and MEM for these fractions if the signals were normalized to total protein levels.

A graphical representation 150 of the relative intensities in the row 148 of the eight protein fractions is also provided. Lastly, UV absorbance spectra 152 of the LB medium-cultured protein fractions 130 (dark gray trace) and MEM medium cultured protein fractions 132 (dashed line) circled in the aforementioned protein rows 144, 148. In this case, corresponding LB and MEM medium-cultured fractions have the same absorption profile. This demonstrates the power and utility of the microarrays, since they can differentiate between different proteins when UV absorbance alone cannot.

### Use as a Forensic Tool

The present invention differentiates between spores that grew naturally (without human intervention) in the environment, for example spores that grew within an animal carcass that died after an anthrax infection, and laboratory-cultured spores. The invention focuses on differentiating among various methods of culture, preparation, storage duration, storage temperature, levels of static electricity in the air, buffer types, ambient conditions such as humidity and air borne particles endemic to certain geographies, and further modifications to spore-formation protocols as opposed to simply identifying biomarkers.

The invented method determines how and where a microorganism was grown, purified, and prepared for dissemination. As such, this results in an enhanced forensic analysis that can contribute to analysis of operator expertise, resources and goals. For example, an operator from an academic environment may adjust certain variables for maximal spore purity, even though not essential for his original purpose. An operator from a military environment is less likely to seek maximally pure preparations but may seek to adjust other parameters in keeping with the standard operating procedures learned in her training. These differences leave recognizable imprints on cultured spore preparations, which are detected by the invention as disclosed, to aid in determining the skill set, experience and resources available to the operator. As such, this invention provides means to identify sources and/or operators of collected spores or molecules.

A myriad of ways are available for initially harvesting field samples. Scooping, wiping, and vacuuming are all mechanical means which are suitable. Adhesion techniques, and electrostatic adsorption are also suitable. Upon harvesting, the harvested material is placed in a container, and the chemical extraction and fractionation techniques discussed supra are utilized.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting, but are instead exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the terms “comprising” and “wherein.” Moreover, in the following claims, the terms “first,” “second,” and “third,” are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “more than” and the like include the number recited and refer to ranges which can be subsequently broken down into subranges as discussed above. In the same manner, all ratios disclosed herein also include all subratios falling within the broader ratio.

One skilled in the art will also readily recognize that where members are grouped together in a common manner, such as in a Markush group, the present invention encompasses not only the entire group listed as a whole, but each member of the group individually and all possible subgroups of the main group. Accordingly, for all purposes, the present invention encompasses not only the main group, but also the main group absent one or more of the group members. The present invention also envisages the explicit exclusion of one or more of any of the group members in the claimed invention.

1. A method for obtaining information about preparation of agent, the method comprising:
a. harvesting agent from a sample;
b. extracting molecules from said agent;
c. fractionating said extracted molecules;
d. generating molecular signatures from said fractions; and
e. comparing said molecular signatures to a library of molecular signatures.

2. The method as recited in claim 1 wherein the sample is harvested from a sample selected from a group consisting of culture medium, laboratory cultures, natural sources, ordnance, other source, and combinations thereof.

3. The method as recited in claim 1 wherein the step of fractionating said extracted molecules for analysis further comprises separating said extracted molecules by their physicochemical properties.

4. The method as recited in claim 1 wherein the step of generating molecular signatures from said fractionated molecules further comprises transferring said fractionated molecules to a biochip by attaching the fractionated molecules to a surface wherein said surface is selected from a group consisting of a matrix of individual molecules, molecular ligands, and adhesive surface.

5. The method as recited in claim 4 wherein the step of generating molecular signatures from said fractionated molecules further comprises comparing the relative abundance of said fractionated molecules.

6. The method as recited in claim 5 wherein the step of comparing the relative abundance of said fractionated molecules comprises reacting said fractioned molecules with fluorescence-tagged detection molecules wherein said detection molecules are selected from a group consisting of antibodies, lectins, macromolecules with functional groups which covalently bond with said fractionated molecules, macromolecules with functional groups which noncovalently bond with said fractionated molecules, and combinations thereof.

7. The method as recited in claim 1 wherein the step of comparing molecular signatures further comprises generating a protein chip to identify molecules with similar physicochemical properties, producing two dimensional heat maps of the protein chip to show the relative abundance of the molecules having similar physicochemical properties, and comparing the two dimensional heat maps heat maps with a two dimensional heat map library of fractions of naturally produced agent.

8. The method as recited in claim 7 wherein said generated protein chip is read using a chip-reader utilizing said conventional software and producing a heat map for comparison to other heat maps characterizing said library of molecular signatures.

9. A method for detecting the source of a sample of bacterial spore of a target bacterium, the method comprising;
a. comparing molecular signatures of the spores of a well known strain of the target bacteria with a molecular signature of the spores of a natural strain of the target bacteria to determine a strain variation pattern; and
b. comparing the strain variation pattern to molecular signatures of the sample to determine features of the molecular signatures of the sample not caused by strain variation.

10. Method as recited in claim 9 wherein the well known strain and the natural strain are adjusted to identical culture techniques.

11. The method as recited in claim 9 wherein molecules associated with features of the molecular signature of the sample not associated with strain variation are confined in cells of a matrix.

12. The method as recited in claim 11 wherein the cells are contacted with affinity tags specific to the molecule.

13. The method as recited in claim 12 wherein each cell is contacted with a predetermined tag.

14. A device for detecting the source of an agent comprising a kit.

15. The device as recited in claim 14 wherein said kit further comprises:
a. a first instrument to harvest samples;
b. a second instrument to extract macromolecules from the harvested samples;
c. a third instrument to fractionate the extracted macromolecules;
d. a fourth instrument and software to generate a molecular signature from said fractionated molecules; and
e. a molecular signature library comprising fingerprints of preparation methods and culture histories, the signature library used to analyze the generated molecular signature.

16. A method for identifying a biomarker, the method comprising:
a. obtaining proteins from a target organism;
b. separating the proteins into a plurality of fractions;
c. isolating the fractions in separate reaction chambers, wherein each of the reaction chambers are arranged in a two-dimensional matrix according to isoelectric point values and hydrophobicity values;
d. contacting each of the fractions with a cocktail of reagents, wherein each of said reagents is capable of binding to less than all of the proteins; and
e. determining which reagents indicate the presence of protein in specific reaction chambers in the matrix.
